(19) United States
(12) Patent Application Publication
(10) Pub. No.: US 2018/0104454 A1
(43) Pub. Date: Apr. 19, 2018

(54) Disc balloon microcatheter

(71) Applicant: Daniel E. Walzman, Bergenfield, NJ (US)

(72) Inventor: Daniel E. Walzman, Bergenfield, NJ (US)

(21) Appl. No.: 15/732,170

(22) Filed: Sep. 26, 2017

Related U.S. Application Data
(60) Provisional application No. 62/496,507, filed on Oct. 19, 2016.

(51) Int. Cl. A61M 25/09; A61B 17/12

(52) U.S. Cl. CPC A61M 25/0905; A61B 17/12113; A61M 2025/09083; A61M 25/09041; A61B 17/12136

(57) ABSTRACT

The present disclosure relates to the field of endovascular treatment. More particularly, the present invention is a disc balloon which is designed to implement safer endovascular treatments, primarily by allowing many middle cerebral artery aneurysms that are currently clipped to be treated with coiling.

### CROSS-REFERENCES

This is a nonprovisional utility application claiming priority to provisional application Ser. No. 62/496,507 filed Oct. 19, 2016 (19 Oct. 2016)

### BACKGROUND OF THE INVENTION

### Field of the Invention

The present disclosure relates to the field of endovascular treatment. More particularly, the present invention is a tool designed to implement an endovascular treatment.

### Background

Middle cerebral artery (MCA) aneurysms and basilar tip aneurysms, among others, often have an unfavorable aneurysm geometry that might limit endovascular therapy. The present invention is a tool to safely and effectively implement an endovascular treatment for many wide neck aneurysms.

### Advantages of the Current Invention

The present invention substantially fulfills the forgoing unmet needs. The present invention would be especially useful for ruptured MCA aneurysms where the distal vessels come off of the aneurysm at acute angles and can be difficult to access with current balloons, which require the operator to cross the aneurysm into the distal vessel. This would make the treatment of many of these aneurysms safer and more effective. It would also allow many MCA aneurysms that are currently clipped to be treated with coiling. It may be advantageous in the treatment of many basilar tip and other wide-neck aneurysms as well.

### BRIEF DESCRIPTION OF THE DRAWINGS

The invention will be better understood and objects other than those set forth above will become apparent when consideration is given to the following detail description thereof. Such description makes reference to the annexed drawing wherein:

FIG. 1 depicts a disc balloon (10) shown in an inflated disc-like shape and disposed within branch vessel (30) and proximate to aneurysm (40). FIG. 1 also depicts micro catheter (20) on which disc balloon is mounted disposed within vessel (32) in cut away, as well as through disc balloon (10) and extending into aneurysm (40). FIG. 1 further depicts radio opaque marker (22). Micro catheter (20) includes a proximal catheter lumen end hole (24) and distal catheter lumen end hole (26) suitable for coil delivery within aneurysm (40).

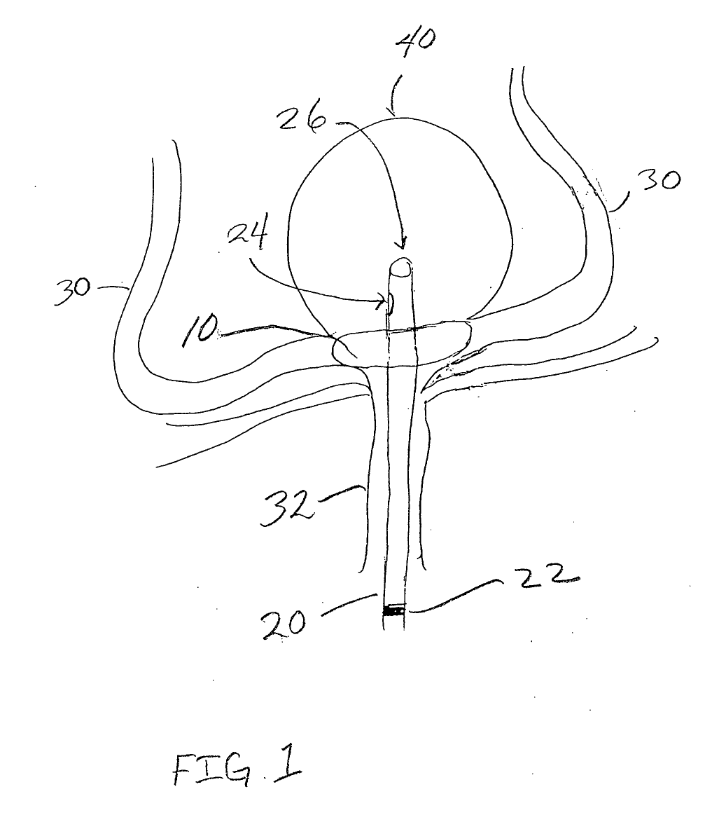

### DETAILED DESCRIPTION OF THE INVENTION

The present disclosure teaches a disc balloon microcatheter device to used to treat wide neck basilar tip, MCA and other aneurysms. In an ideal situation this could be a Scepter (MicroVention)-like balloon or a design akin to the HyperGlide™ (Medtronic), but with a disc shaped balloon only 1 mm-3 mm in length and 3 mm-6 mm in diameter. The length of the balloon should not exceed its diameter.

The device can have its catheter tip extending beyond the distal end of the balloon, typically extending 0.5 mm-20 mm. In a preferred embodiment, the lengths vary from approximately 1 mm, 3.5 mm, and 6 mm. In order to accommodate different sized aneurysms, it can vary from 0 cm to 2 cm.

The primary designed use of the balloon taught is to coil through the main balloon catheter, with an appropriate radio-opaque marker 3 cm from the distal tip on that catheter, which lines up with the detachment marker on neuro-coils. In another embodiment of the present invention, the “Scepter”-like version, contains three lumens (channel within a tube): one for a balloon, and two extending through the balloon and into the aneurysm.

The more proximal lumen ends 0 mm-10 mm distal to the distal end of the balloon, and more distal lumen ends 0.5 mm-20 mm more distal than the first. In the “HyperGlide”-like design a similar effect is achieved with only two lumens. One lumen would only be used for coil delivery, and either of the other two lumens, which both end distal to the distal end of the disc-balloon, then serves a dual function for coil delivery as well as balloon inflation and deflation.

The catheter of this invention is composed of a first lumen that has a diameter that is larger proximal to said balloon than distal to said disc balloon, wherein the distal diameter is sized so that an appropriately sized wire will be occlusive of that segment when inserted, thus allowing delivery of fluid through the proximal segment of a catheter to inflate said disc balloon.

The dual-use lumen has a lumen diameter slightly larger proximal to the disc balloon than distal to the disc balloon. When a wire, coil, or coil-pusher wire of an appropriately sized diameter is in the lumen and extending into the more narrow segment of the microcatheter beyond the distal end of the disc-balloon, it would be occlusive in the distal segment, so when contrast or other fluid is injected proximally it would flow into the balloon but not beyond the balloon, thus inflating said balloon.

Similarly, for the lowest profile device, a single lumen microcatheter can be used with a “HyperGlide” like design as just described, wherein the single lumen can act to both inflate the balloon and deliver coils. However, the limitation of such a single lumen design is that when a wire is removed from the lumen, as is common practice between delivery of coils, the balloon will deflate, whether such deflation is desirable at that juncture or not.

There are two classically described techniques to treat/coil wide necked aneurysms without a stent. One is balloon-assisted coiling. The second is “dual microcatheter technique,” whereby two microcatheters are advanced into the aneurysm. The first coil can be placed through one microcatheter, but not detached until additional coil(s) are placed through the other microcatheter, which help anchor the first coil in place. This current disc balloon microcatheter design allows simultaneous use of both techniques, while never having to cross the aneurysm, and only having to catheterize the aneurysm once, reducing the potential risk associated with crossing the aneurysm as well as the risk of rupturing the aneurysm each time a catheter is advanced into it.

It will be understood that the above particular embodiment is shown and described by way of illustration only. The principles and the features of the present disclosure may be employed in various and numerous embodiments thereof without departing from the scope and spirit of the disclosure as claimed. The above-described embodiment illustrated the scope of the disclosure but does not restrict the scope of the disclosure.

What is claimed is:

1. A catheter mounted with a disc shaped balloon located approximately 0 cm to 3 cm near the distal tip, to facilitate balloon-assisted coiling of a wide-neck aneurism with a single device, said disc shaped balloon having a diameter equal to or greater than its length.

2. The catheter of claim 1, wherein a first lumen is used for fluid delivery to inflate balloon and at least a second lumen facilitates delivery of coils.

3. The catheter of claim 2, wherein at least one of the coil-delivery lumena end at the distal end hole of the catheter, to allow delivery of a guiding wire through the distal end of the microcatheter.

4. The catheter of claim 1, wherein a first lumen is used for fluid delivery to inflate said disc balloon, at least a second lumen with an end-hole 0 mm-30 mm from the distal end of said disc balloon, facilitates delivery of coils, and at least a third lumen that facilitates delivery of coils terminating approximately 0 mm to 35 mm distal to the distal end of said disc balloon proximal to said aneurysm.

5. The catheter of claim 4, wherein at least one of the coil-delivery lumena end at the distal end hole of the catheter, to allow delivery of a guiding wire through the distal end of the catheter.

6. The catheter of claim 1, wherein a first lumen has a diameter that is larger proximal to said balloon than distal to said disc balloon, wherein the distal diameter is adapted for an appropriately sized wire.

7. The catheter of claim 6, wherein at least one of the coil-delivery lumena end at the distal end hole of the catheter, to allow delivery of a guiding wire through the distal end of said catheter.

8. The catheter of claim 6, with an additional lumen that facilitates delivery of coils distal to said disc balloon.

9. The catheter of claim 8, wherein at least one of the coil-delivery lumena end at the distal end hole of the catheter, to allow delivery of a guiding wire through the distal end of said catheter.

10. The catheter of claim 6, with at least two additional lumens that facilitate delivery of coils to said disc balloon.

11. The catheter of claim 10, wherein at least one of the coil-delivery lumena end at the distal end hole of the catheter, to allow delivery of a guiding wire through the distal end of said catheter.
